(19) United States
(12) Patent Application Publication
(10) Pub. No.: US 2024/0398821 A1
(43) Pub. Date: Dec. 5, 2024

(54) FLAVONOID DERIVATIVES WITH GABAA RECEPTOR ACTIVITY AND METHODS OF USE

(71) Applicant: RUTGERS, THE STATE UNIVERSITY OF NEW JERSEY, New Brunswick, NJ (US)

(72) Inventors: Eileen Carry, New Brunswick, NJ (US); James E. Simon, New Brunswick, NJ (US); Jacques Roberge, Piscataway, NJ (US)

(21) Appl. No.: 18/699,103

(22) PCT Filed: Oct. 6, 2022
(86) PCT No.: PCT/US2022/045904

§ 371 Date: Apr. 5, 2024

Related U.S. Application Data
(60) Provisional application No. 63/253,341, filed on Oct. 7, 2021.

(51) Int. Cl. A61K 31/536; A61K 9/00; A61K 31/352; A61K 31/517; A61K 45/06; A61P 25/20; A61P 25/22; A61P 25/32; C07D 239/91; C07D 265/22; C07D 311/32

(52) U.S. Cl. CPC A61K 31/536; A61K 9/0053; A61K 31/352; A61K 31/517; A61K 45/06; A61P 25/20; A61P 25/22; A61P 25/32; C07D 239/91; C07D 265/22; C07D 311/32

(57) ABSTRACT

Provided herein are GABA_{A}R positive modulator compounds of Formula I and methods for treatment of anxiety, distress and/or alcohol use disorder using these compounds. In certain embodiments, the compounds are stable and efficacious flavonoid derivatives that do not exhibit negative side effect(s) when administered in conjunction with alcohol consumption.

### CROSS-REFERENCE TO RELATED APPLICATION

This application claims priority to U.S. Provisional Patent Application No. 63/253,341 entitled “FLAVONOID DERIVATIVES WITH GABAA RECEPTOR ACTIVITY AND METHODS OF USE,” filed Oct. 7, 2021, the disclosure of which is incorporated herein by reference in its entirety.

### BACKGROUND

Benzodiazepines (BZDs) are a class of GABA_{A}R positive modulators, and the most commonly prescribed acute anxiolytic agent, with an estimated 5.2% of the population having an active prescription to one or more BZD. BZDs are a standard of treatment for acute alcohol withdrawal, although their outpatient use is unsafe for individuals with a history of alcohol use disorder.

BZDs are highly addictive and are commonly abused. While BZD use rarely leads to fatal overdoses when taken alone, BZDs are highly toxic when combined with other CNS depressants, such as ethanol. A major risk of BZDs is their synergistic and additive effects when combined with ethanol. Thus, while GABA_{A}R positive modulators have demonstrated significantly utility and efficacy as acute anxiolytics, their potential toxicity if combined with ethanol limits their use, can lead to expensive hospital stays, and overdose fatalities. Another major issue is that prescribers often do not know if patients are honest about their alcohol consumption.

Currently, 70% of the population consumes alcohol. Dihydromyricetin (DHM), a natural flavonoid, is a GABA_{A}R positive allosteric modulator. DHM has been shown to reduce intoxicating effects of ethanol in rats and mice and is currently sold as a dietary supplement. This is promising towards the development of a safer GABA_{A}R PAM, without ethanol enhancing effects. However, DHM lacks drug-like properties, leading to inadequate CNS bioavailability necessary for reliable translation to clinical efficacy.

Therefore, there is an urgent need for clinically efficacious compounds that are GABA_{A}R positive modulators and that also lack any ethanol-related enhancement of side effects. The present disclosure addresses this need.

### BRIEF SUMMARY

In one aspect, a compound of Formula I, or a salt, solvate, tautomer, enantiomer, diastereoisomer, and/or isotopically labeled derivative thereof is provided:

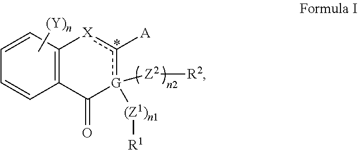

wherein:
   [symbol] is independently at each occurrence a single or double bond, wherein the X—C* bond and the G-C* bond are selected such that:
      if the X—C* bond is a double bond, then
         the G-C* bond is a single bond, and
         X is N, or
      if the X—C* bond is a single bond, then
         G-C* bond is a single or a double bond, and
         X is O, N—R, or S;
   each occurrence of Y is independently R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl;
   G is C or N,
      wherein if G is N or if the G-C* bond is a double bond, then Z² and R² are absent;
   A is C₆₋₁₀ aryl or C₅₋₁₀ heteroaryl, each of which is optionally substituted by 1 to 5 substituents independently selected from the group consisting of R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂;
   Z¹ and Z² are independently at each occurrence O, CH₂, CHF, or CF₂;
   R¹ and R² are independently at each occurrence OR, SR, or R,
      or —(Z¹)ₙ₁—R¹ is H,
      or —(Z²)ₙ₂—R² is H;
   each occurrence of R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl;
   n is 1, 2, 3, 4, or 5;
   n1 is 1, 2, or 3; and
   n2 is 1, 2, or 3.

In certain embodiments, compounds of Formula I, including pharmaceutical compositions of the compound of Formula I, are useful in treating, ameliorating, and/or preventing alcohol use disorder (AUD) in a subject in need thereof.

### BRIEF DESCRIPTION OF THE FIGURES

The drawings illustrate generally, by way of example, but not by way of limitation, various embodiments of the present application.

FIG. 1 is shows a structure of DHM (dihydromyricetin).

FIG. 2A shows a cryo-EM structure of diazepam (valium) in the BZD binding site (PDB:6X3X). His102 interactions are associated with al selectivity.

FIG. 2B shows a cryo-EM structure of flumazenil in the BZD binding site with lowest energy conformation of DHM (PDB:6T6U).

FIG. 3A shows a docked pose of dihydromyricetin.

FIG. 3B shows an overlap of docking results for dihydromyricetin and myricetin.

FIG. 4A shows a docked pose of a compound of Formula I (C1).

FIG. 4B shows an overlap of docking conformations of DHM and a compound of Formula I (C1).

FIG. 4C shows an overlap of docked conformations of 6-bromoflavone and a compound of Formula I (C1).

FIGS. 5A-5B show concentration response curve representing reported extrapolated EC₅₀ value of EC2-118 (FIG. 5B), in comparison to that of allopregnanolone (a full GABA_{A}R PAM and GABA_{A}R agonist, FIG. 5A).

FIG. 6 shows a HPLC-UV-MS/MS analysis of EC2-118.

FIG. 7 is a representation of rat movements during an open field test. Increased time in the open arms signifies anti-anxiety activity. At the 3.95 mg/kg dose we observed that the rat spend much more time exploring the open arms demonstrating a significant reduction of anxiety compared to the vehicle arm of the assay. The purple lines represent the motion of the rat.

FIG. 8 shows a Rotarod test with EC2-118 at 3.95 and 12.5 mg/kg IP in comparison to vehicle control.

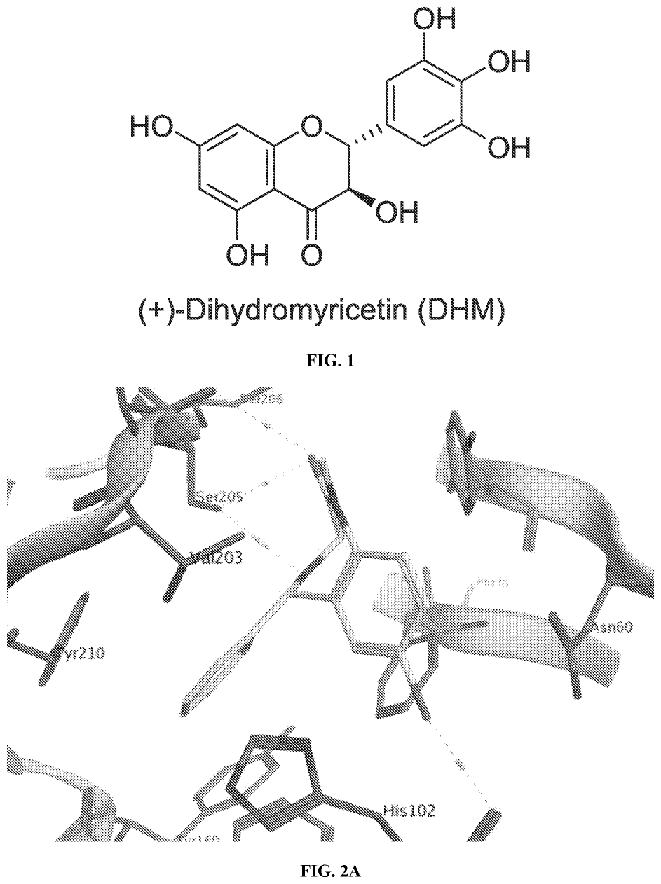

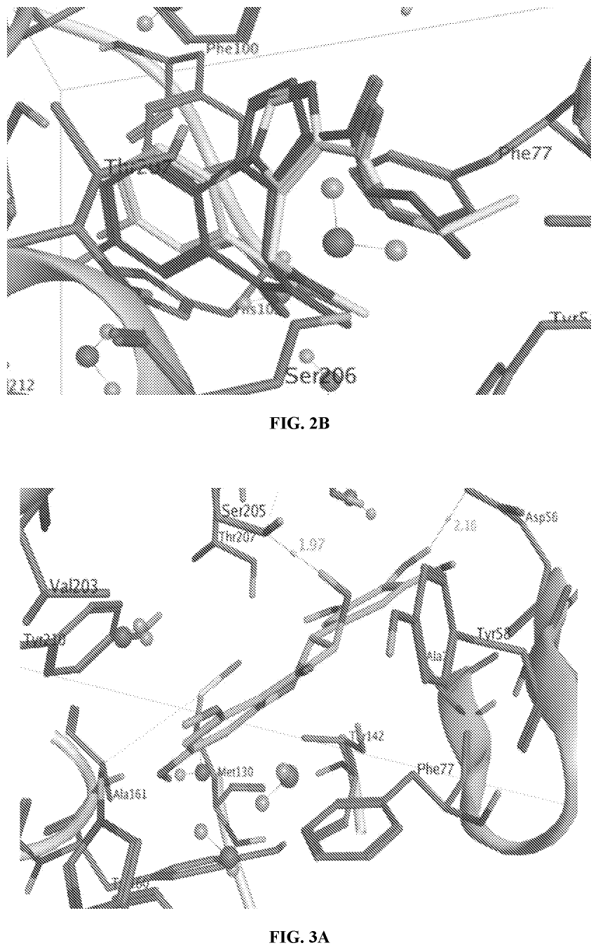

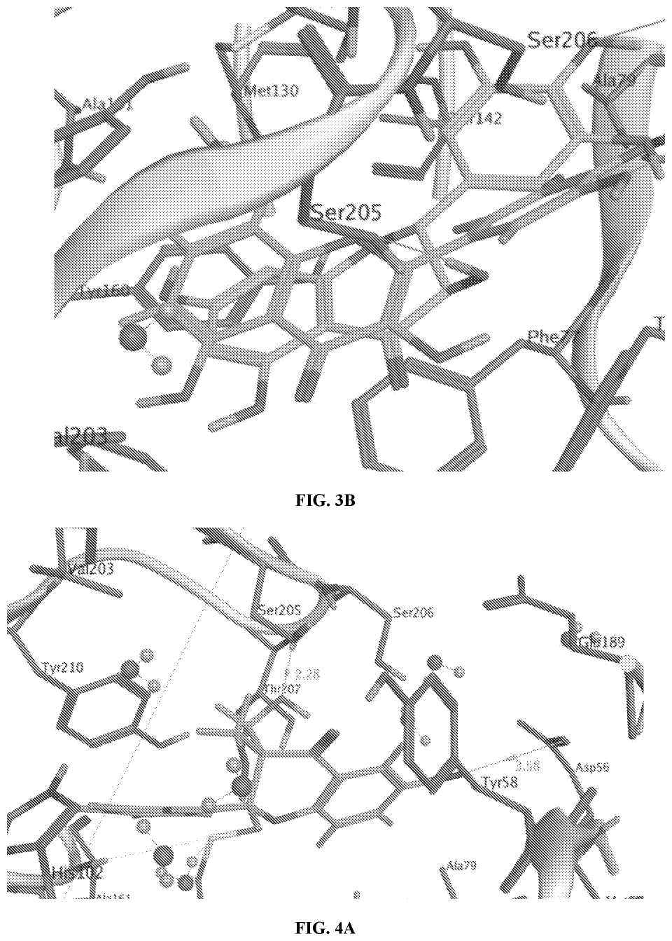

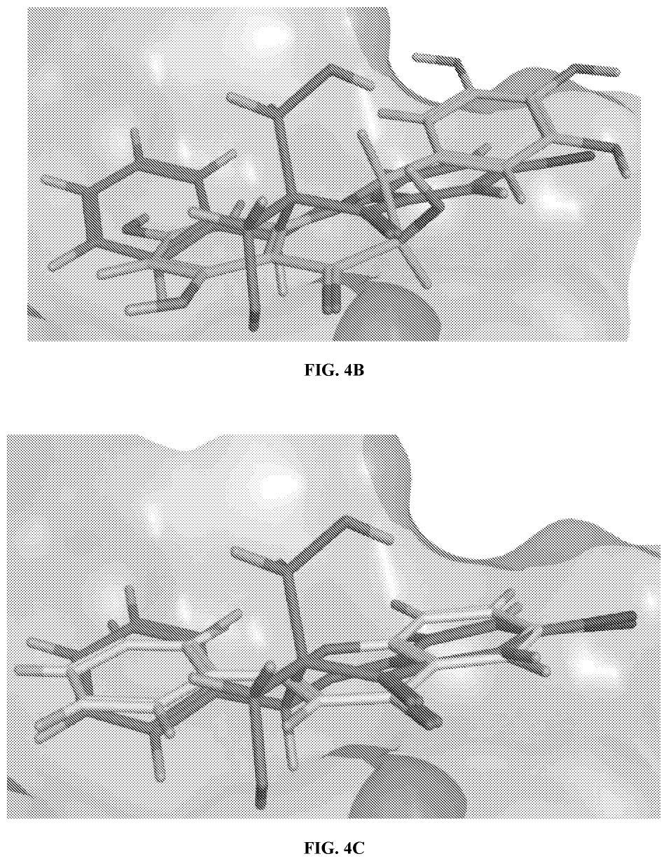

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and/or C(═NOR)R, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl.

Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “halocycloalkyl” as used herein refers to a cycloalkyl group that has one or more C—H bonds replaced by C—X bond, where X is a F, Cl, Br, or I atom. When all such hydrogen atoms are replaced by a halogen, the halocycloalkyl is a perhalogenated halocycloalkyl. For example, and without limitation, a perfluorinated cycloalkyl. Mixed halogen (more than one time of halogen atom) halocycloakyls are also contemplated.

The term “heterocycloalkyl” as used herein refers to a cycloalkyl group in which one or more of the carbon atoms are replaced by a heteroatom such as, B, O, N, S, or P, as well as stable oxides of these heteroatoms. Heterocycloalkyl groups can also contain one or more degrees of unsaturation, such as carbon-carbon double bonds or carbon-heteroatom double bonds. Heterocycloalkyl groups can be optionally substituted by one or more of any of the substituents described herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. The term heterocyclyl includes rings where a CH₂ group in the ring is replaced by one or more C═O groups, such as found in cyclic ketones, lactones, and lactams. Examples of heterocyclyl groups containing a C═O group include, but are not limited to, 3-propiolactam, γ-butyrolactam, δ-valerolactam, and ε-caprolactam, as well as the corresponding lactones. A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. A heterocyclyl ring designated C_{x-y} can be any ring containing ‘x’ members up to ‘y’ members, including all intermediate integers between ‘x’ and ‘y’ and that contains one or more heteroatoms, as defined herein. In a ring designated C_{x-y}, all non-heteroatom members are carbon. Heterocyclyl rings designated C_{x-y} can also be polycyclic ring systems, such as bicyclic or tricyclic ring systems. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15-28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

As used herein, the term “efficacy” refers to the maximal effect (Eₘₐₓ) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

The abbreviations of reagents used herein have the following definitions:
TPAP tetrapropylammonium perruthenate;
NMO N-methyl morpholine-N-oxide;
THE tetrahydrofuran;
TIPSCl triisopropylsilyl chloride; and
DCE dichloroethane.

### Preparation of Compounds

Compounds of Formula I or otherwise described herein can be prepared by the general schemes described herein, using the synthetic method known by those skilled in the art. The following examples illustrate non-limiting embodiments of the compound(s) described herein and their preparation.

In various embodiments, a compound of Formula I, or a salt, solvate, tautomer, enantiomer, diastereomer, and/or isotopically labeled derivative thereof, has the structure:

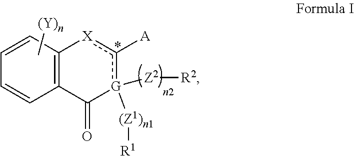

wherein:
   [symbol] is independently at each occurrence a single or double bond, wherein the X—C* bond and the G-C* bond are selected such that:
      if the X—C* bond is a double bond, then
         the G-C* bond is a single bond, and
         X is N, or
      if the X—C* bond is a single bond, then
         G-C* bond is a single or a double bond, and
         X is O, N—R, or S;
   each occurrence of Y is independently R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl;
   G is C or N,
      wherein if G is N or if the G-C* bond is a double bond, then Z² and R² are absent;
      A is C₆₋₁₀ aryl or C₅₋₁₀ heteroaryl, each of which is optionally substituted by 1 to 5 substituents independently selected from the group consisting of R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂;
   Z¹ and Z² are independently at each occurrence O, CH₂, CHF, or CF₂;
   R¹ and R² are independently at each occurrence OR, SR, or R,
      or —(Z¹)ₙ₁—R¹ is H,
      or —(Z²)ₙ₂—R² is H;
   each occurrence of R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl;
   n is 1, 2, 3, 4, or 5;
   n1 is 1, 2, or 3; and
   n2 is 1, 2, or 3.

The X—C* bond and G-C* bond refer to the bond between variable X or G, respectively, and the carbon marked with an * in the structure of Formula I.

In various embodiments, a compound of Formula I, or a salt, solvate, tautomer, enantiomer, diastereomer, and/or isotopically labeled derivative thereof, has the structure:

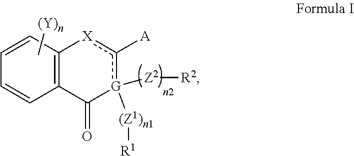

wherein:
   [symbol] is independently at each occurrence a single or double bond;
   each occurrence of Y is independently R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl;
   X is O, N, N—R, or S;
   G is C or N, wherein if G is N, then Z² and R² are absent;
   A is C₆₋₁₀ aryl or C₅₋₁₀ heteroaryl, each of which is optionally substituted by 1 to 5 substituents independently selected from the group consisting of R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂;
   Z¹ and Z² are independently at each occurrence H, O, CH₂, CHF, or CF₂;
   R¹ and R² is are each independently absent, H, OR, SR, or R;
   each occurrence of R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl;
   n is 1, 2, 3, 4, or 5;
   n1 is 1, 2, or 3; and
   n2 is 1, 2, or 3.

In certain embodiments, the compound does not comprise any O—O bond. In certain embodiments, the compound does not comprise any S—O bond (such as in —S—O—). In certain embodiments, the compound does not comprise any S—S bond. In certain embodiments, the compound does not comprise any S—S—S bond.—

In various embodiments, X is O. In various embodiments, X is S. In various embodiments, X is NR. In various embodiments, X is NH. In various embodiments, G is C.

In various embodiments, Z¹ and Z² are CH₂. In various embodiments, n1 and n2 are 1. In various embodiments, n1 and n2 are 2. In various embodiments, n1 and n2 are 3. In various embodiments, R¹ and R² are OH. In various embodiments, A is optionally substituted phenyl. In various embodiments, A is phenyl or 3-nitrophenyl. In various embodiments, G is N, Z¹ is H, and R¹ is absent.

In various embodiments, when A is phenyl, it is not substituted with OH or F at the location of substituent Q:

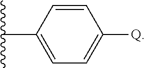

In various embodiments, the compound of Formula I has one of the following structures:

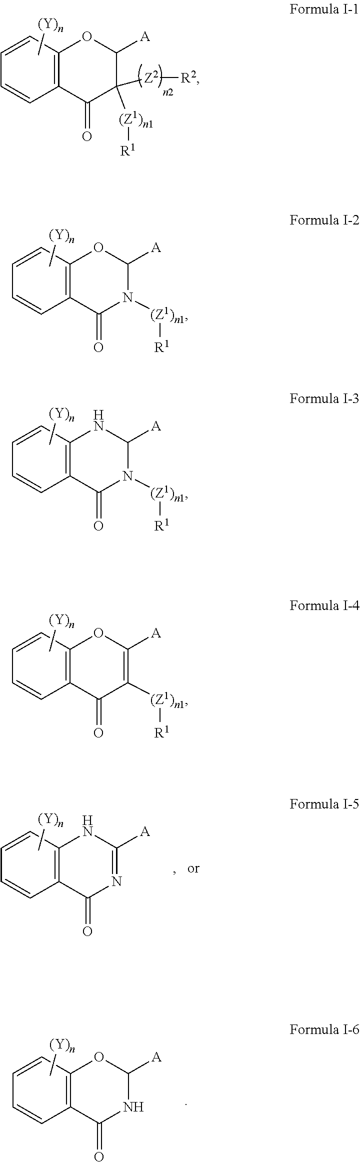

In various embodiments, the compound of Formula I has one of the following structures:

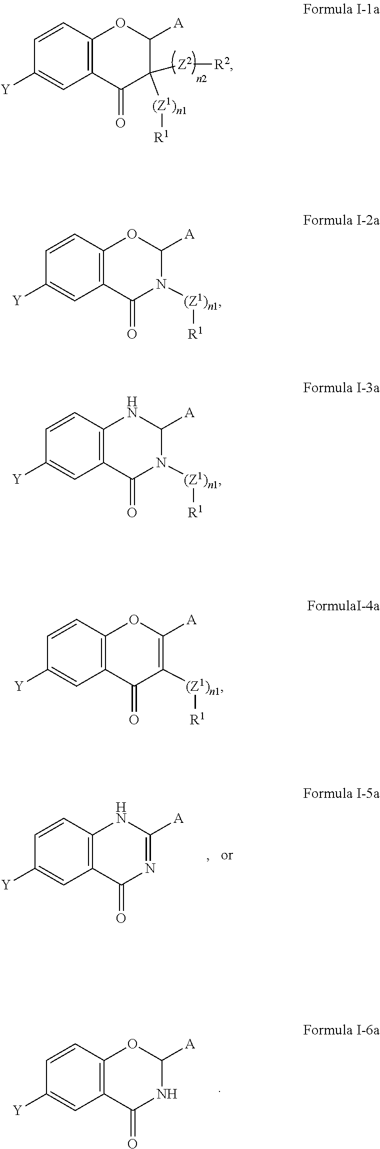

In various embodiments, the ring in the compound of Formula I bearing one or more Y substituents can have any one of the following substitution patterns:

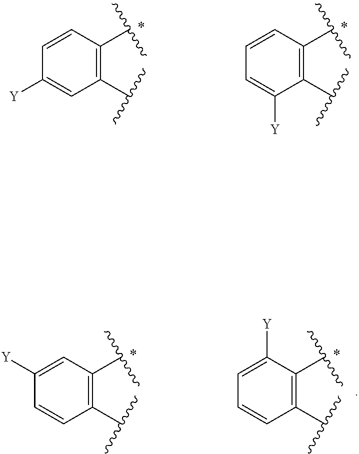

Wavy lines represent the remainder of the rings and substituents in the compound of Formula I. The wavy line marked with a * is bonded to variable X.

In various embodiments, the compound of Formula I can present as an enantiomerically pure composition of one of the following enantiomers:

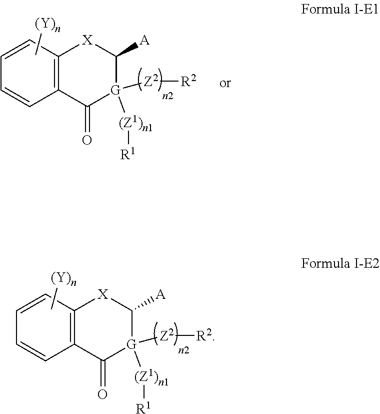

If multiple chiral centers are present in the compound of Formula I, then the compounds of Formula I-E1 and I-E2 represent diastereomers.

When the carbon to which the A substituent is connected to is the only chiral center in the compound of Formula I, the precise stereochemical designation of that center (R or S) will depend on the definitions of the variables in the compound of Formula I-E1 or Formula I-E2. The enantiomeric purity of the composition can be at equal to or greater than about 99.5, 99, 98, 97, 96, 95, 94, 93, 92, 91, 90, 85, 80, 75, 70, 65, or 60% Formula I-E1 or the enantiomeric purity of the composition can be at equal to or greater than about 99.5, 99, 98, 97, 96, 95, 94, 93, 92, 91, 90, 85, 80, 75, 70, 65, or 60% Formula I-E2. The compound of Formula I can also be present as a racemic mixture.

In various embodiments, A is:

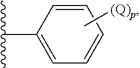

wherein p is an integer from 1 to 5; and
Q is selected from the group consisting of F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂, wherein R is independently selected from the group consisting of hydrogen, C₁₋₆ alkyl, C₁₋₆ haloalkyl, C₃₋₈ cycloalkyl, C₃₋₈ halocycloalkyl, and combinations thereof. In various embodiments, R in A is H, methyl, ethyl, propyl, or isopropyl.

In various embodiments, p is 1. In various embodiments, p is 2. In various embodiments, p is 3. In various embodiments, p is 4. In various embodiments, p is 5.

In various embodiments, A is:

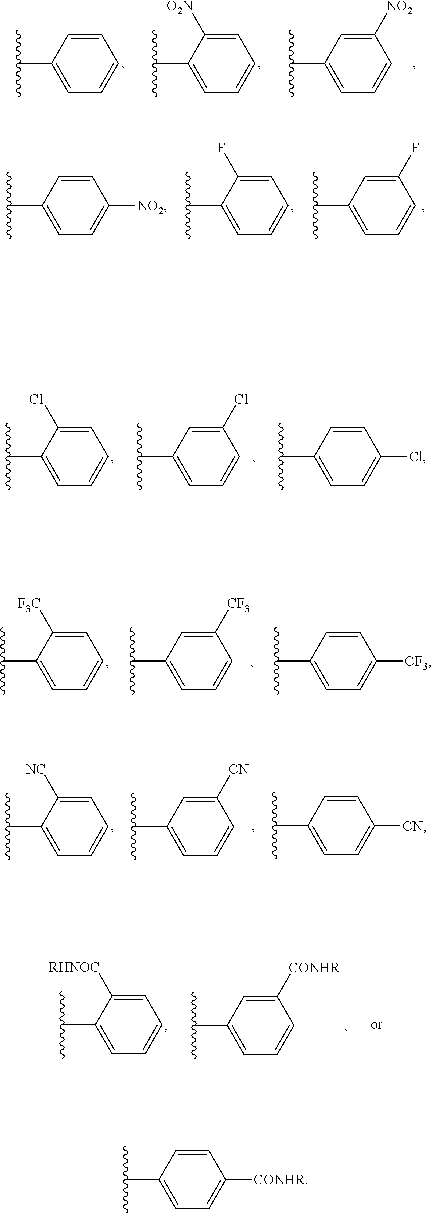

Any of the A groups described herein can be combined with the compound of Formula I.

In various embodiments, the compound of Formula I is selected from the group consisting of:

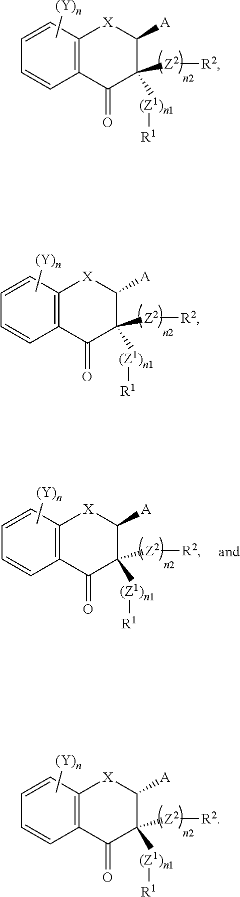

In various embodiments, each Y is independently selected from R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl, wherein R is independently at each occurrence hydrogen, C₁₋₆ alkyl, C₁₋₆ haloalkyl, C₃₋₈ cycloalkyl, C₃₋₈ halocycloalkyl, or combinations thereof. In various embodiments, at each occurrence R is independently H, methyl, ethyl, n-propyl, iso-propyl, n-butyl, sec-butyl, or t-butyl.

In various embodiments, Y is halogen (F, Cl, Br, or I). In various embodiments, Y is Br. In various embodiments, the compound of Formula I, or tautomer thereof, is selected from the group consisting of:

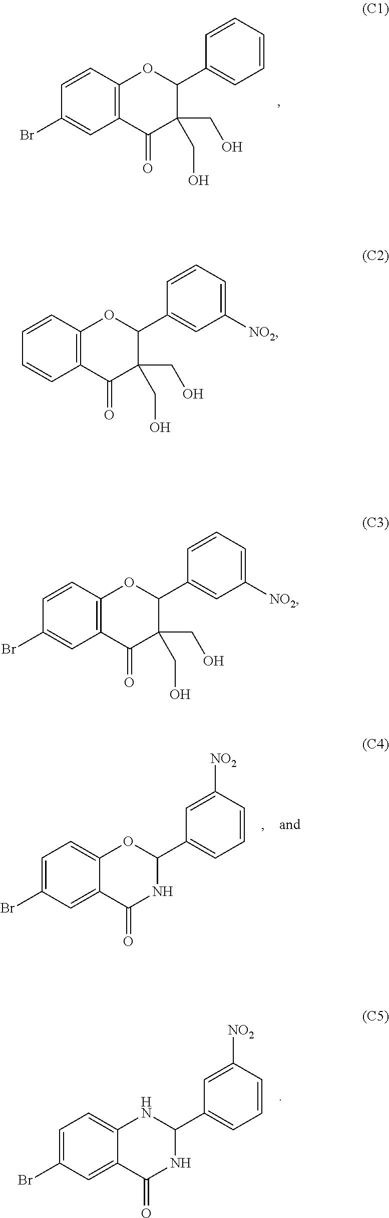 [SMILES: O=C1c2cc(Br)ccc2OC(c2ccccc2)C1(CO)CO ; O=C1c2ccccc2OC(c2cccc([N+](=O)[O-])c2)C1(CO)CO ; O=C1c2cc(Br)ccc2OC(c2cccc([N+](=O)[O-])c2)C1(CO)CO ; O=C1NC(c2cccc([N+](=O)[O-])c2)Oc2ccc(Br)cc21 ; O=C1NC(c2cccc([N+](=O)[O-])c2)Nc2ccc(Br)cc21]

Tautomers of compounds of Formula I include, for example, tautomers of compounds C4 and C5, which have the structures:

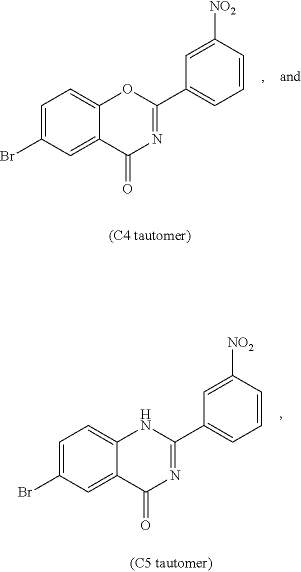 [SMILES: O=c1nc(-c2cccc([N+](=O)[O-])c2)oc2ccc(Br)cc12 ; O=c1nc(-c2cccc([N+](=O)[O-])c2)[nH]c2ccc(Br)cc12]

The compound of Formula I can be formulated in a pharmaceutical composition containing the compound of Formula I and at least one pharmaceutically acceptable carrier or excipient as described herein.

In various embodiments, Compounds of Formula I can be synthesized according to Scheme 1, 2, 3, or 4

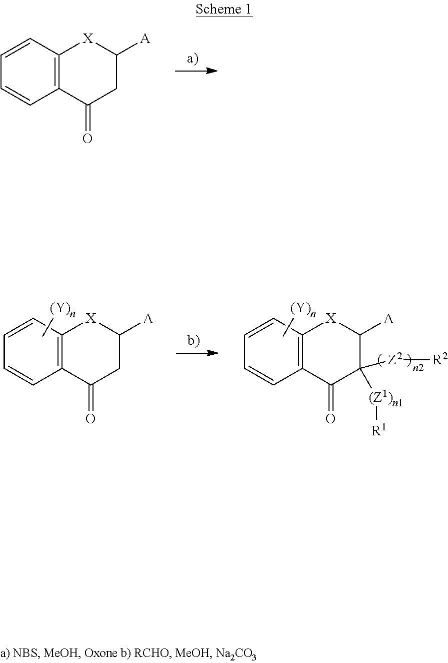

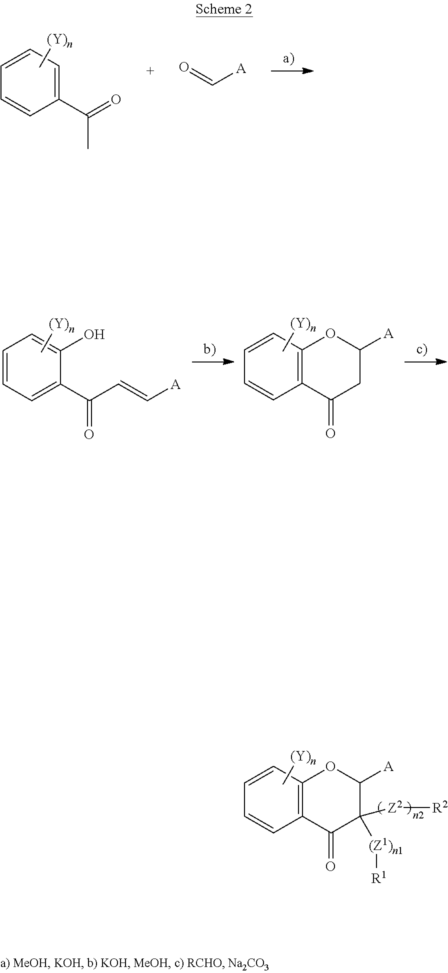

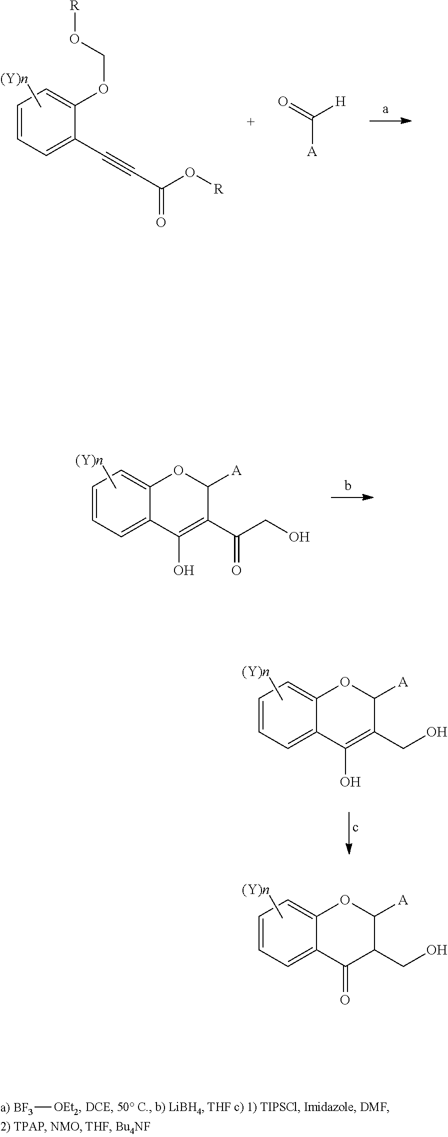

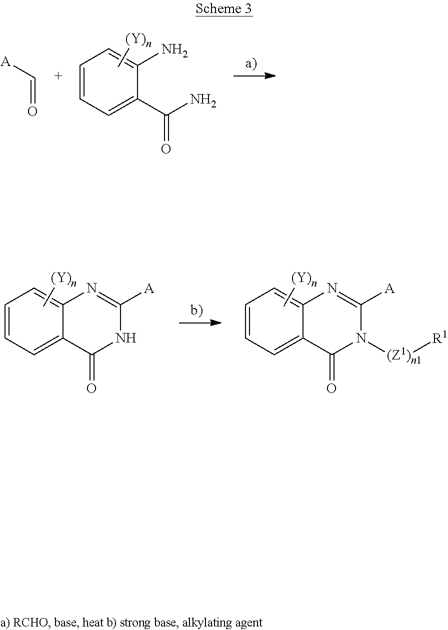

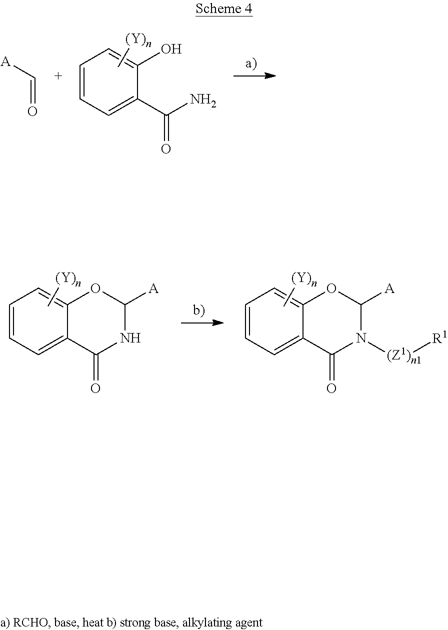

In various embodiments, the compound of Formula I crosses the blood-brain barrier.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means.

Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl, triisopropylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable. The silyl group are also selectively removable with fluoride ions.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

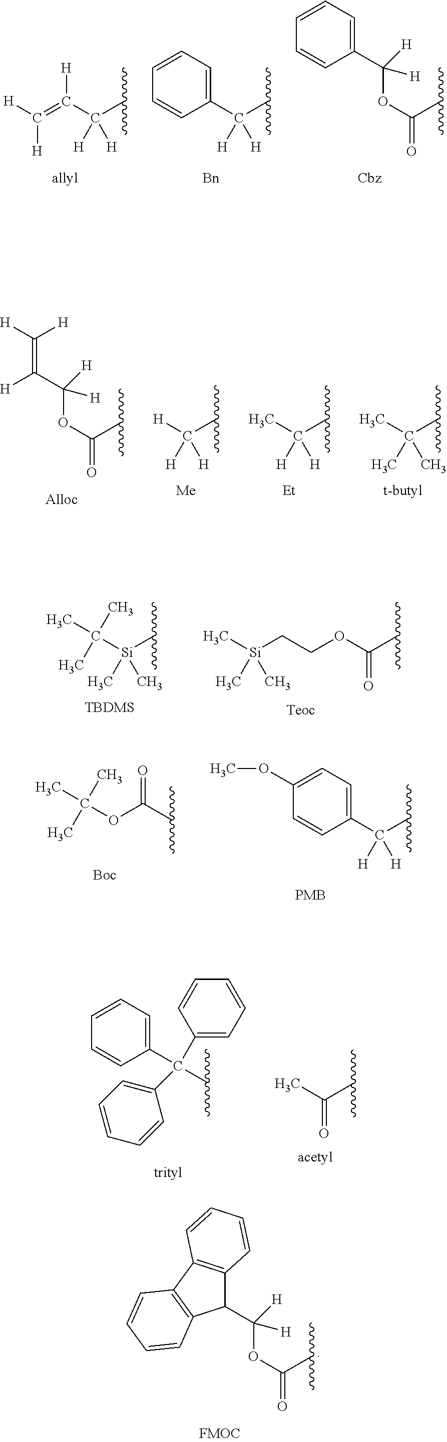

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

Methods of Treatment, Amelioration, and/or Prevention

The disclosure includes a method of treating, ameliorating, and/or preventing alcohol use syndrome (AUD) using the compounds of Formula I. The disclosure also includes a method of treating, ameliorating, and/or preventing anxiety and distress using the compounds of Formula I. The disclosure also includes a method of treating, ameliorating, and/or preventing benzodiazepine physical dependence (also known as anxiolytic use disorder).

Treatment of AUD typically requires pharmacotherapies during early abstinence to address the decreased GABAergic transmission. BZDs are the most commonly prescribed GABA_{A}R PAMs but enhance respiratory depression and inebriating effects of alcohol and opiates, leading to many fatal accidents and overdoses. Dihydromyricetin (DHM) is a natural flavonoid and a GABA_{A}R PAM/inhibitor of ethanol-induced GABA_{A}R potentiation, with some bioactivity for AUD treatment. However, as with most flavonoids, DHM lacks drug-like properties, leading to inadequate, variable bioavailability.

Surprisingly and unexpectedly, in various embodiments, subjects taking both a compound of Formula I and consuming alcohol (ethanol), will not suffer any serious and/or detrimental side effects as a result of taking the compound of Formula I. In various embodiments, administration of the compound of Formula I results in therapeutically efficacious concentration of the compound in the brain as a result of the compound of Formula I crossing the blood-brain barrier (BBB).

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent(s) that treats AUD and/or anxiety and/or distress.

In certain embodiments, administering the compound(s) described herein to the subject allows for administering a lower dose of the additional therapeutic agent as compared to the dose of the additional therapeutic agent alone that is required to achieve similar results in treating AUD, anxiety or distress in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are co-formulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating, ameliorating, and/or preventing AUD, anxiety and/or distress. These additional therapeutic agents may comprise compounds that are commercially available or synthetically accessible to those skilled in the art. These additional therapeutic agents are known to treat or reduce the symptoms of AUD or anxiety.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a AUD, anxiety or distress. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, may be carried out using known procedures, at dosages and for periods of time effective to treat AUD or anxiety in the patient. An effective amount of the therapeutic compound necessary to achieve a therapeutic effect may vary according to factors such as the state of the disease or disorder in the patient; the age, sex, and weight of the patient; and the ability of the therapeutic compound to treat AUD or anxiety in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of AUD and/or anxiety in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, nano-encapsulations, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%0, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10, 1%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release, slow release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of AUD or anxiety in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### Example 1: Synthesis of 6-bromo-3,3-bis(hydroxymethyl)-2-phenylchroman-4-one

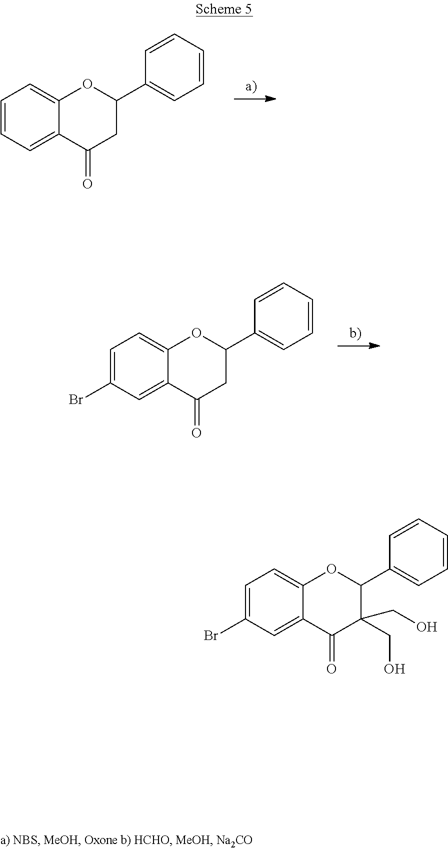 [SMILES: O=C1CC(c2ccccc2)Oc2ccccc21 ; O=C1CC(c2ccccc2)Oc2ccc(Br)cc21 ; O=C1c2cc(Br)ccc2OC(c2ccccc2)C1(CO)CO]

### 6-bromo-3,3-bis(hydroxymethyl)-2-phenylchroman-4-one

2-phenyl-2,3-dihydrochromen-4-one (223 mg, 1.0 mmol) was dissolved in 1:1 acetone:water (10 mL). Oxido hydrogen sulfate (152 mg, 1.0 mmol, 1 equiv.) and NaBr (206 mg, 2.0 mmol, 2 equiv.) was added and reaction stirred for 36 hrs. at room temperature. The reaction was then quenched with water and extracted with ethyl acetate (2×50 mL) and dried under vacuum. The final product was isolated with silica gel flash column chromatography, with a solvent gradient of 0-10% Ethyl acetate and hexane, to yield 6-bromo-3,3-bis(hydroxymethyl)-2-phenylchroman-4-one (95 mg, 0.31 mmol, 31% yield). 6-bromo-3,3-bis(hydroxymethyl)-2-phenylchroman-4-one was dissolved in acetonitrile (10 mL). Na2CO3 (59 mg, 0.56 mmol, 4 equiv) and formaldehyde (37% w/v aq., 0.5 mL, 0.56 mmol, 4 equiv.) were added and the reaction stirred at room temperature for 48 hours. The reaction was then quenched with water, extracted into ethyl acetate, dried with Na₂SO₄, filtered, and dried under vacuum. The final product was isolated with a C-18 flash column, and a solvent gradient of 5-60% acetonitrile and water with 0.1% formic acid, to yield C1 (21.3 mg, 0.05 mmol, 26% yield). ¹H-NMR (500 MHz, DMSO-d₆) δ 2.94 (dd, 1H, J=5.9, 4.7 Hz), 3.14 (dd, 1H, J=5.9, 5.2 Hz), 3.61 (dd, 1H, J=5.9, 5.2 Hz), 4.14 (dd, 1H, J=5.9, 4.7 Hz), 4.65 (t, 1H, J=5.2 Hz), 4.99 (s, 1H), 5.81 (s, 1H), 7.06 (d, 1H, J=8.8 Hz), 7.34-7.41 (m, 3H) 7.52 (d, 2H, J=7.5 Hz), 7.71 (dd, 1H, J=6.2, 2.7 Hz) 7.84 (d, 1H, J=2.6 Hz). ¹³C-NMR (500 MHz, DMSO-d₆) δ 55.9, 59.8, 82.8, 113.5, 121.1, 122.8, 128.1, 128.5, 129.1, 129.2, 135.9, 138.5, 160.1, 193.2.

### Example 2: Synthesis of 3,3-bis(hydroxymethyl)-2-(3-nitrophenyl)chroman-4-one

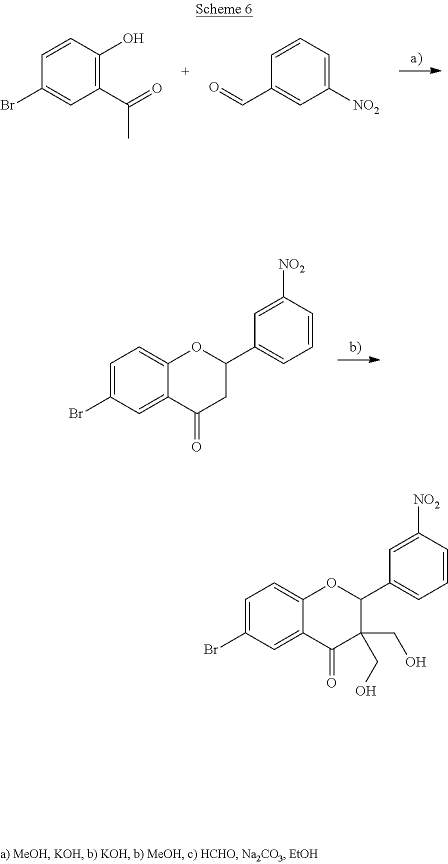 [SMILES: CC(=O)c1cc(Br)ccc1O ; O=Cc1cccc([N+](=O)[O-])c1 ; O=C1CC(c2cccc([N+](=O)[O-])c2)Oc2ccc(Br)cc21 ; O=C1c2cc(Br)ccc2OC(c2cccc([N+](=O)[O-])c2)C1(CO)CO]

1-(5-bromo-2-hydroxyphenyl)ethan-1-one (1 equiv., 951 mg, 4.4 mmol) was dissolved in 1:1 THE to ethanol. 5 equiv. of NaOH (2M) was slowly added and the reaction stirred at room temperature for 15 minutes. 3-nitrobenzaldehyde (1.2 equiv., 798 mg, 5.3 mmol) was added and the reaction stirred at room temperature for 48 hrs. The reaction was then quenched with water, extracted into ethyl acetate, and dried under vacuum. 3-nitro-2-phenylchromen-4-one was isolated via silica gel column chromatography with a solvent gradient of 10-25% ethyl acetate to hexane (yield=45%). 3-nitro-2-phenylchromen-4-one (104 mg, 0.3 mmol) was then dissolved in ethanol (10 mL). Na₂CO₃ (59 mg, 0.56 mmol, 4 equiv.) and formaldehyde (37% w/v aq., 0.5 mL, 0.56 mmol, 4 equiv.) were added and reaction stirred at room temperature for 48 hours. The reaction was then quenched with water, extracted into ethyl acetate, dried with Na₂SO₄, filtered, and dried under vacuum. The final product was isolated silica gel column, with a solvent gradient of 10-25% ethyl acetate to hexane (52 mg, 0.13 mmol, 56% yield). ¹H-NMR (500 MHz, DMSO-d₆) δ 8.41 (t, J=2.0 Hz, 1H), 8.25 (dd, J=7.9, 2.3 Hz, 1H), 8.00 (d, J=7.7 Hz, 1H), 7.87 (d, J=2.6 Hz, 1H), 7.78-7.70 (m, 2H), 7.12 (d, J=8.8 Hz, 1H), 5.99 (s, 1H), 5.16 (t, J=4.6 Hz, 1H), 4.76 (t, J=5.1 Hz, 1H), 4.16 (dd, J=10.9, 4.8 Hz, 1H), 3.49 (dd, J=11.2, 5.1 Hz, 1H), 3.20 (dd, J=11.2, 5.2 Hz, 1H), 2.92 (dd, J=10.9, 4.6 Hz, 1H).

### Example 3: Synthesis of 6-bromo-2-(3-nitrophenyl)-2,3-dihydroquinazolin-4(1H)-one

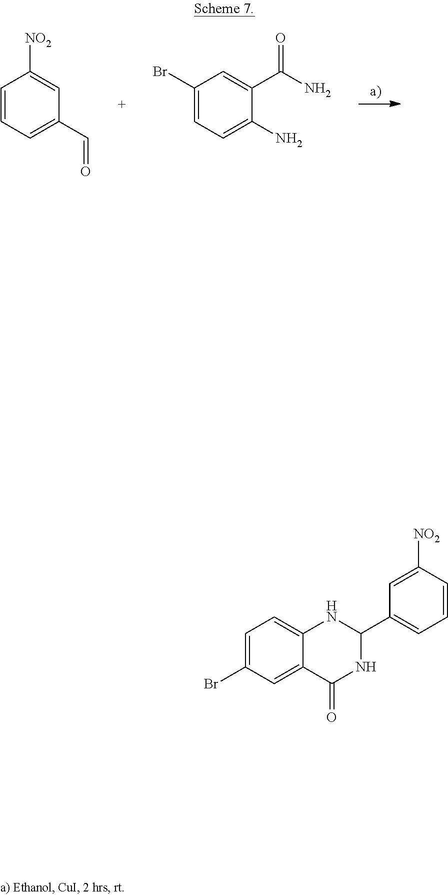 [SMILES: O=Cc1cccc([N+](=O)[O-])c1 ; NC(=O)c1cc(Br)ccc1N ; O=C1NC(c2cccc([N+](=O)[O-])c2)Nc2ccc(Br)cc21]

6-bromo-2-(3-nitrophenyl)-2,3-dihydroquinazolin-4(1H)-one 3-nitrobenzaldehyde (1.2 equiv., 109 mg, 0.7 mmol) and 2-amino-5-bromobenzamide (1 equiv., 130 mg, 0.6 mmol) were dissolved in ethanol (10 mL) with a catalytic amount of CuI (˜1 mg) and stirred at room temperature for 2 hrs. The reaction was then diluted with water, extracted into ethyl acetate, and dried under vacuum. 6-bromo-2-(3-nitrophenyl)-2,3-dihydroquinazolin-4(1H)-one (C5) was isolated via silica gel column chromatography with a solvent gradient of 25-100% ethyl acetate to hexane. Final product afforded a yellow crystalline material (Yield=39%, 82 mg, 0.2 mmol). ¹H NMR (500 MHz, DMSO-d₆) δ 8.68 (s, 1H), 8.32 (s, 1H), 8.23-8.17 (m, 1H), 7.90 (d, J=7.8 Hz, 1H), 7.69 (t, J=8.0 Hz, 1H), 7.65 (d, J=2.5 Hz, 1H), 7.56 (s, 1H), 7.40 (ddd, J=8.7, 2.4, 0.9 Hz, 1H), 6.79-6.71 (m, 1H), 5.97 (s, 1H).

### Example 4: Synthesis of 6-bromo-2-(3-nitrophenyl)-2,3-dihydro-4H-benzo[e][1,3]oxazin-4-one

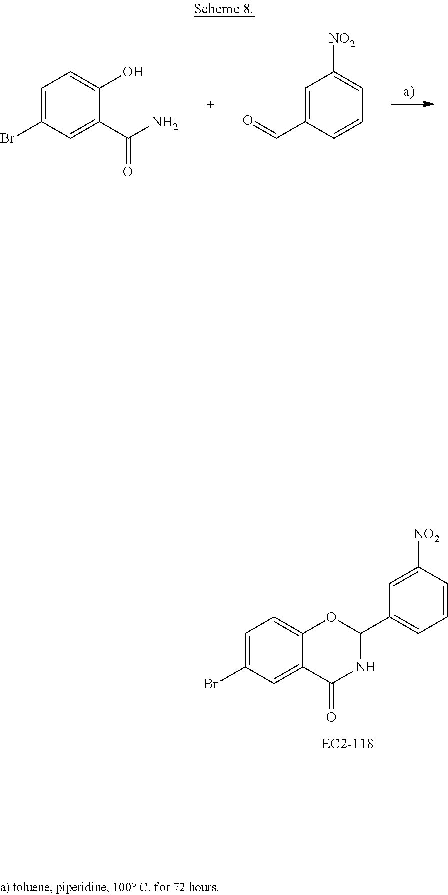 [SMILES: NC(=O)c1cc(Br)ccc1O ; O=Cc1cccc([N+](=O)[O-])c1 ; O=C1NC(c2cccc([N+](=O)[O-])c2)Oc2ccc(Br)cc21]

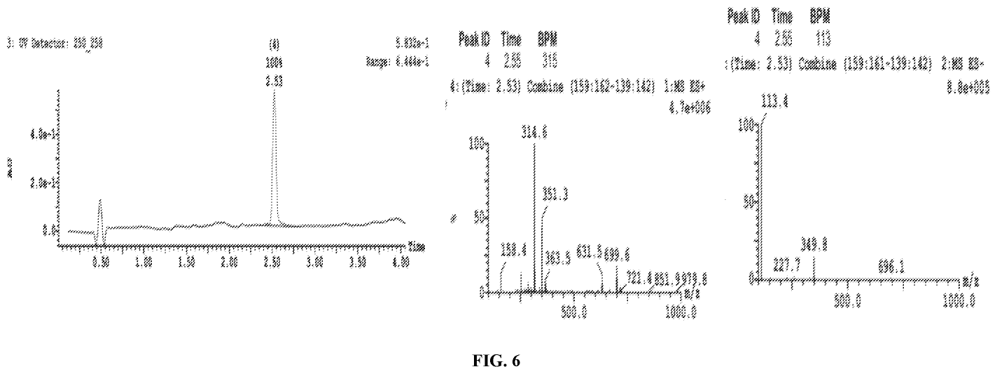

EC2-118 crystallized out of solution and was filtered to obtain an isolated yield of 30%. H NMR (500 MHz, DMSO-d₆) δ 9.30 (s, 1H), 8.39 (t, J=1.9 Hz, 1H), 8.30 (dd, J=8.0, 1.7 Hz, 1H), 8.03 (d, J=7.8 Hz, 1H), 7.86 (d, J=2.5 Hz, 1H), 7.76 (t, J=8.0 Hz, 1H), 7.70 (dd, J=8.7, 2.6 Hz, 1H), 7.10 (d, J=8.7 Hz, 1H), 6.63 (d, J=1.8 Hz, 1H). FIG. 6 shows the HPLC-UV-MS/MS analysis of EC2-118.

### 6-bromo-2-(3-nitrophenyl)-2,3-dihydro-4H-benzo[e][1,3]oxazin-4-one

3-nitrobenzaldehyde (1.5 equiv., 1200 mg, 7.9 mmol) and 5-bromo-2-hydroxybenzamide (1 equiv., 1150 mg, 5.3 mmol) were dissolved in toluene (100 mL). Piperidine (0.35 equiv., 1.3 mmol) was added and the reaction refluxed at 100° C. for 6 hrs. The reaction was then diluted with water, extracted with ethyl acetate, and dried under vacuum. The final product was isolated via silica gel column chromatography with a solvent gradient of 25-100% ethyl acetate to hexane, affording a white crystalline product (Yield=29%, 550 mg, 1.6 mmol). ¹H NMR (500 MHz, DMSO-d₆) δ 9.30 (s, 1H), 8.39 (t, J=1.9 Hz, 1H), 8.30 (dd, J=8.0, 1.7 Hz, 1H), 8.03 (d, J=7.8 Hz, 1H), 7.86 (d, J=2.5 Hz, 1H), 7.76 (t, J=8.0 Hz, 1H), 7.70 (dd, J=8.7, 2.6 Hz, 1H), 7.10 (d, J=8.7 Hz, 1H), 6.63 (d, J=1.8 Hz, 1H).

### Example 5: Screening for Intrinsic GABA_{A}R Activity

As part of the screening model, a total of 7 molecules were analyzed, including (R)-Dihydromyricetin (natural flavanol), 6-bromo-3′-nitroflavone (commercially available synthetic flavone), and compounds of molecules (EC2-52, EC2-104, EC2-117, EC2-118, and EC2-124), Scheme 9. The inclusion of 6-bromo-3′-nitrofavone—a known potent competitive binder to the BZD binding site of GABAARs with low intrinsic efficacy, was made to confirm this activity.

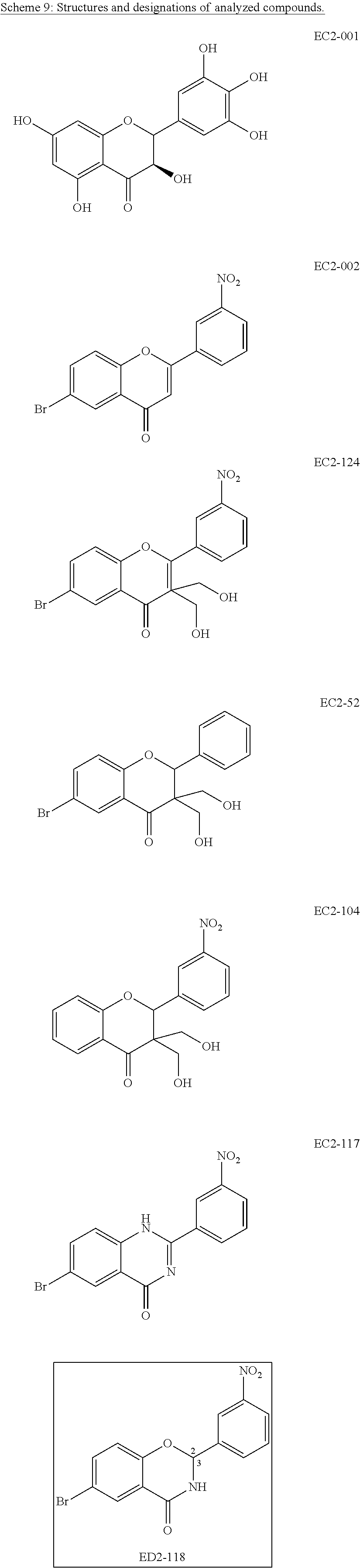

The compounds in Scheme 9 also have the following alternative designations herein. EC2-52 (Compound C1), EC2-104 (Compound C2), EC2-117 (Compound 5 and tautomers thereof), EC2-118 (Compound 4 and tautomers thereof), and EC2-124 (Compound C3).

Electrophysiology studies in Xenopus oocytes expressing subtype specific GABAA receptors Electrophysiology studies were conducted in Xenopus oocytes expressing α5β3γ2 GABAARs. Xenopus oocytes were stored in incubation media (pH 7.5), consisting of ND96 supplemented with 2 mM sodium pyruvate, 0.1 nM gentamycin, and 10 mL heat inactivated HyClone horse serum. Stage 4 to 5 oocytes were injected with 40 nL of cDNA coding for a5, 03, and 72 subunits in a ratio of 1:1:10, utilizing a Drummond Nanoject III. Injected oocytes were stored at 18° C. and used in electrophysiology studies within 2-7 days of injection. Whole cell two-electrode voltage clamp (TEVC) recording were conducted on oocytes in the presence of GABA. Oocytes were voltage clamped at a membrane potential of −70 mV. The oocyte recording chamber was continuously perfused with modified bath solution (MBS) with select compound (10 μM) and GABA (10 μM), with a final concentration of 1% DMSO. It has been previously demonstrated that this concentration of DMSO did not significantly affect GABA_{A}R function. Oocytes were perfused at a rate of 3 mL/min. Electrophysiology recordings were conducted in triplicate compound for each compound. The change of GABA_{A}R activity was determined by comparison of GABA_{A}R potentiation to the EC₂₀ with control saline in the presence of GABA. Statistical significance was determined. Statistical significance of electrophysiology results compound GABAergic modulation was determined using a paired t-test (Vehicle control vs. Compound, GraphPad software, San Diego CA, USA). p<0.05 was considered significant.

As an assessment for potential anxiolytic activity, compounds were screened for in vitro GABAA receptor activity utilizing electrophysiology studies. This is a well-established method of assessing activity of GABAA receptor positive allosteric modulators (PAMs) with known anxiolytic activity. The link between anxiolytic and sedative activity associated with α1/2/3βγ GABA_{A}R subtypes is also well-established by previous studies. and there is a well-established history of clinical efficacy, utility, and risks associated with the use of full GABA_{A}R PAMs [i.e. benzodiazepines (BZDs)] in the treatment of anxiety and seizure disorders.

To evaluate GABAA receptor activity, compounds were evaluated with electrophysiology studies of against 4 GABAA subtypes (GABAA α1β2γ2L, α2θ3γ2L, α5θ3γ2L and α4θ3δ) in positive allosteric modulator (PAM) mode using the SyncroPatch automated platform at room temperature. A 6-point concentration-response curve of each test compound (10, 1, 0.1, 0.01, 0.001, or 0.0001 μM in DMSO). The rapid addition protocol was used, in which compound is rapidly applied and then washed off from the cell. 0.3% DMSO was used as negative control.

The assay was conducted as follows:

### Concentrations and Replicates

Test compounds: 6-point concentration-response curve of each test compound (10, 1, 0.1, 0.01, 0.001, 0.0001 μM in DMO).

Positive control: 5-point concentration-response curve of allopregnanolone (10, 1, 0.1, 0.01, 0.01 PM).

Negative control: 0.3% DMSO

Each compound is tested across the plate with a single concentration per cell. A minimum of 2 wells are obtained per each concentration.

### Storage:

Powder stocks and DMSO stocks are stored at room temperature (in the dark). The powder stocks are diluted to 3 mM in 100% DMSO on the day of experiment.

### Solutions:

SyncroPatch specific solutions are used throughout the assay. DMSO remains constant at 0.3% in the external solution throughout the assay.

### Application Protocol:

The rapid addition protocol is used, in which compound is rapidly applied and then washed off from the cell. To test for PAM activity, the agonist GABA EC10-20 is applied three times (with wash steps in-between) as a control and to show activation reproducibility, followed by a 1 to 2 minute pre-incubation of client compound and then re-application of GABA EC10-20 in the presence of client compound. After a further wash step, maximum GABA (10 mM) is applied.

### Analysis:

Automated patch clamp-recordings are performed using the SyncroPatch 384i. The voltage protocol generation and data collection are performed with PatchController384 V1.9.0 and Data Controller V1.9.0.

The fold increase is generated using the following equation, (I_{comp}/I_{control})−1, where I_{comp} is the current amplitude in the presence of the compound and I_{control} is the current amplitude in the presence of agonist alone. This builds an EC₅₀ concentration-response curve, where 0 represents no PAM activity and >0 represents PAM activity. If a “bell-shape curve” is observed at the higher concentrations, showing a reduction in activity in comparison to previous concentrations, then to allow for a more accurate curve fit, these concentrations are removed from the graphs where necessary and noted in the compound table. The % Eₘₐₓ relative to Allopregnanolone is generated using the following equation,
[(I_{MaxComp}/I_{AveMaxCtrl})*100], where I_{MaxComp} is the individual maximum fold increase in current for each compound and I_{AveMaxCtrl} is the average maximum fold increase generated in the presence of the control, allopregnanolone.

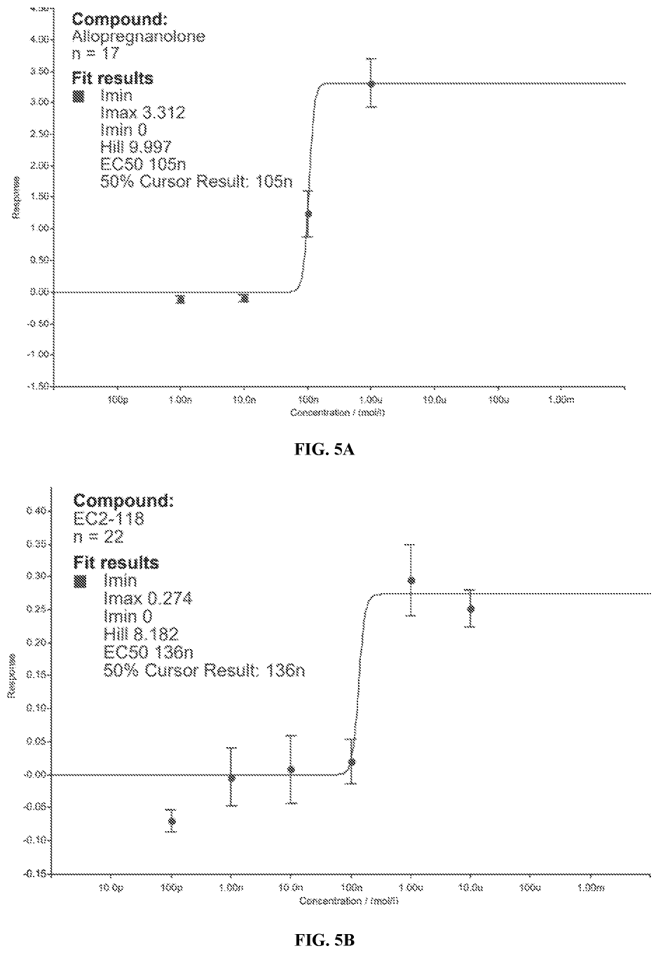

To test for PAM activity, the agonist GABA EC 10-20 was applied three times (with wash steps in-between) as a control and to show activation reproducibility, followed by a 1 to 2 minutes pre-incubation of client compound and then re-application of GABA EC 10-20 in the presence of client compound. After a further wash step, maximum GABA (10 mM) was applied. Allopregnanolone was utilized as a positive control. FIGS. 5A and 5B show for dose response of EC2-118 in comparison to that of allopregnanolone (positive control).

TABLE 1
Electrophysiology results for compounds EC2-52, EC2-104, EC2-117, EC2-118, and EC2-124.

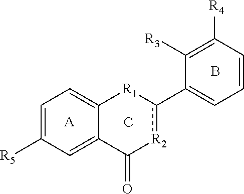

| | | | | | | | Preliminary Classification of type of |
|---|---|---|---|---|---|---|---|
| Cpd. | R₁ | R₂ | R₃ | R₄ | R₅ | Potencyª | GABAAR modulation |
| EC2-52 | O | 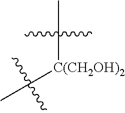 | H | H | Br | α1βy (N.R.) α2βy (N.R.) α5βy (1,920* +22%) α4βδ (N.R.) | Partial α5 GABAAR PAM |
| EC2-104 | O | 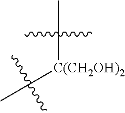 | H | NO₂ | H | α1βy (N.R.) α2βy 11,800* (+28%) α5βy (N.R.) α4βy > 10,000 (+30%) | Partial α2 GABAAR PAM |
| EC2-117 | NH | 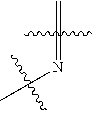 [SMILES: *N=*] | H | NO₂ | Br | α1 βy (N.R.) α2βy (N.R.) α5βy (N.R.) α4ßy (N.R.) | Not active |
| EC2-118 | O | 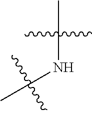 [SMILES: *N*] | H | NO₂ | Br | α1βy (136*) (+29%) αβy (125*) +26%) α5βy (N.R.) α4βy (N.R.) | Partial α1/2 GABAAR PAM |
| EC2-124 | O | 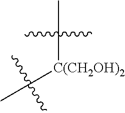 | H | NO₂ | Br | α1ßy (N.R.) α2βy (N.R.) α5βy (N.R.) α4βy (N.R.) | Not Active |
| 6- bromo- 3′- nitroflavone (EC2- 002) | O | 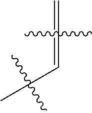 [SMILES: *C=*] | H | NO₂ | Br | α1βy 5.9* (−21%) α2βy N.R. α5βy N.R. α4βy > 10,000* (−30%) | Partial α1 NAM |

^{a}represented as *EC50 or *IC50 (nM). Subtype specific data is provided if available.

### Example 6: In Vivo Anxiolytic and Sedative Activity with IP Administration

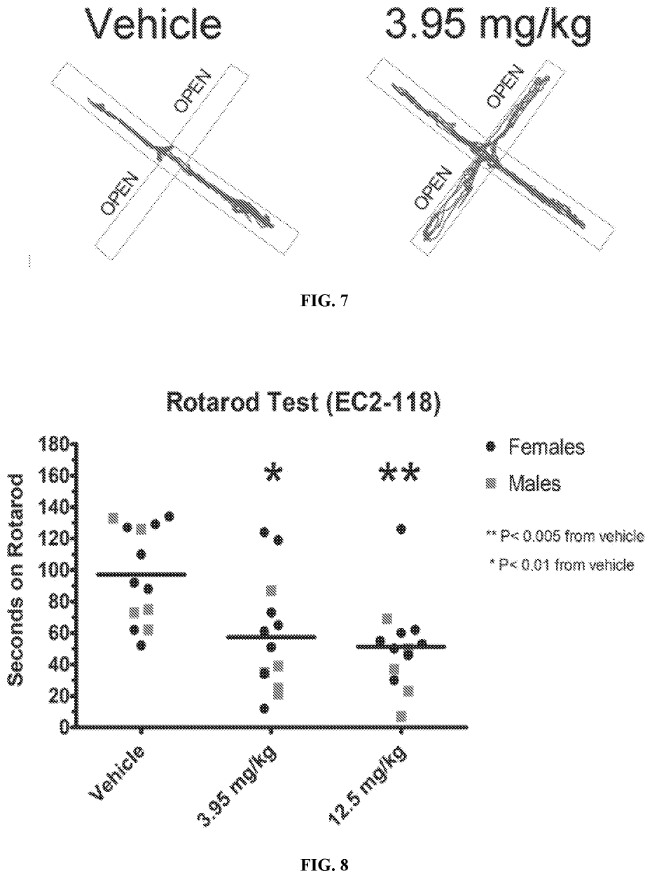

In vivo studies in rats were conducted in the Bello laboratory as preliminary screening for activity of EC2-118. Male and female rats were exposed to an open field test (OFT) and elevated plus maze (EPM) 7 days apart (Kshatriya et al., 2020). Each group (n=8/group, total=24) received IP injection 30 min prior to IP dosing of EC2-118 or vehicle control. We performed OFT and EPM in 52 adult SD rats (26 male/26 female). The groups were as follow vehicle (0.9% Saline, 4% Tween 80, and 4% DMSO; n=9 males; n=10 females), 3.95 mg/kg (n=10 males; n=8 females), 12.5 mg/kg (n=7 males, females n=8). Statistical analysis was completed using TWO-ANOVA with Treatment and Sex as factors and showed significant differences between 3.95 mg/kg dose and vehicle control (FIG. 7).

Rats were given EC2-118 (3.95 mg/kg IP) and alcohol (3 g/kg) and assessed for duration of loss-of-righting-reflex (LORR). RUEC2-118 (3.95 mg/kg, 12.95 IP) did not induce an LORR. Further, EC2-118 3.95 mg/kg did not have a significant impact on alcohol-induced LORR. The effects of EC2-118 (12.5 mg/kg IP) on alcohol-induced LORR were not determined.

In summary, compounds of Formula I displayed statistically significant anxiolytic effects at a dose of 3.95 mg/kg IP in male and female rats. A dose of 12.5 mg/kg EC2-118 IP did not exhibit a significant effect in the open field test (OFT), but the observation of increased lethargy at 12.5 mg/kg IP coupled with enhanced motor impairment at 12.5 mg/kg IP (as evidenced by Rotorod studies), suggest a dose-dependent increase in sedative effects, as is expected with GABA_{A}R PAMs.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following enumerated embodiments are provided, the numbering of which is not to be construed as designating levels of importance:
Embodiment 1 provides compound of Formula I, or a salt, solvate, tautomer, enantiomer, diastereoisomer, and/or isotopically labeled derivative thereof:

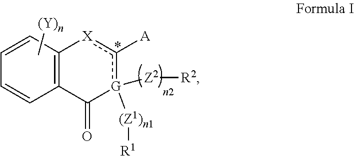

wherein:
   [symbol] is independently at each occurrence a single or double bond, wherein the X—C* bond and the G-C* bond are selected such that:
      if the X—C* bond is a double bond, then
         the G-C* bond is a single bond, and
         X is N, or
      if the X—C* bond is a single bond, then
         G-C* bond is a single or a double bond, and
         X is O, N—R, or S;
   each occurrence of Y is independently R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl;
   G is C or N,
      wherein if G is N or if the G-C* bond is a double bond, then Z² and R² are absent;
   A is C₆₋₁₀ aryl or C₅₋₁₀ heteroaryl, each of which is optionally substituted by 1 to 5 substituents independently selected from the group consisting of R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂;
   Z¹ and Z² are independently at each occurrence O, CH₂, CHF, or CF₂;
   R¹ and R² are independently at each occurrence OR, SR, or R,
      or —(Z¹)ₙ₁—R¹ is H,
      or —(Z²)ₙ₂—R² is H;
   each occurrence of R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl;
   n is 1, 2, 3, 4, or 5;
   n1 is 1, 2, or 3; and
   n2 is 1, 2, or 3.

Embodiment 2 provides the compound of embodiment 1, which is:

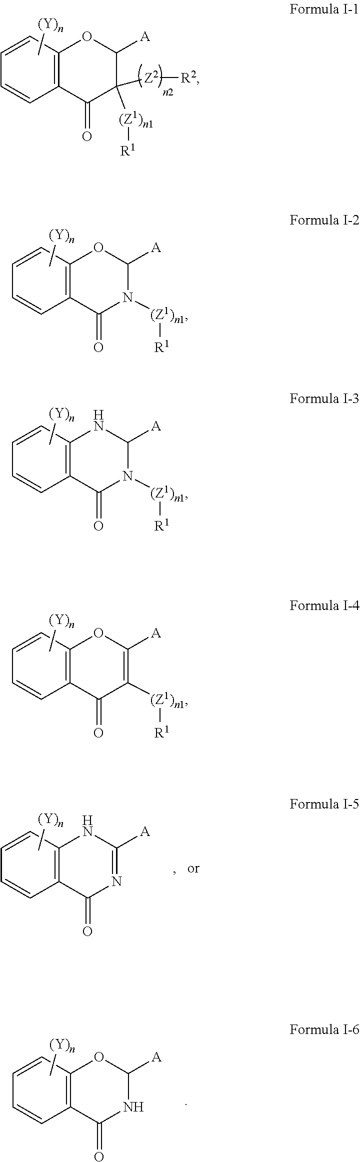

Embodiment 3 provides the compound of any one of embodiments 1-2, which is

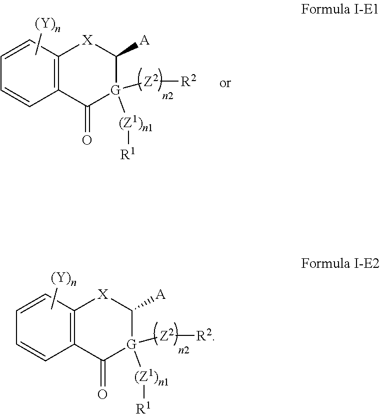

Embodiment 4 provides the compound of any one of embodiments 1-3, wherein X is 0.

Embodiment 5 provides the compound of any one of embodiments 1-4, wherein G is C.

Embodiment 6 provides the compound of any one of embodiments 1-5, wherein Z¹ and Z² are CH₂.

Embodiment 7 provides the compound of any one of embodiments 1-6, wherein n1 and n2 are 1.

Embodiment 8 provides the compound of any one of embodiments 1-7, wherein R¹ and R² are OH.

Embodiment 9 provides the compound of any one of embodiments 1-8, wherein A is

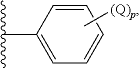

wherein
   p is an integer from 1 to 5;
   Q is selected from the group consisting of F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂; and
   R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl.

Embodiment 10 provides the compound of any one of embodiments 1-9, wherein Y is F, Cl, Br, or I.

Embodiment 11 provides the compound of any one of embodiments 1-10, wherein Y is Br.

Embodiment 12 provides the compound of any one of embodiments 1-11, wherein G is N, Z¹ is H, and R¹ is absent.

Embodiment 13 provides the compound of any one of embodiments 1-12, wherein A is

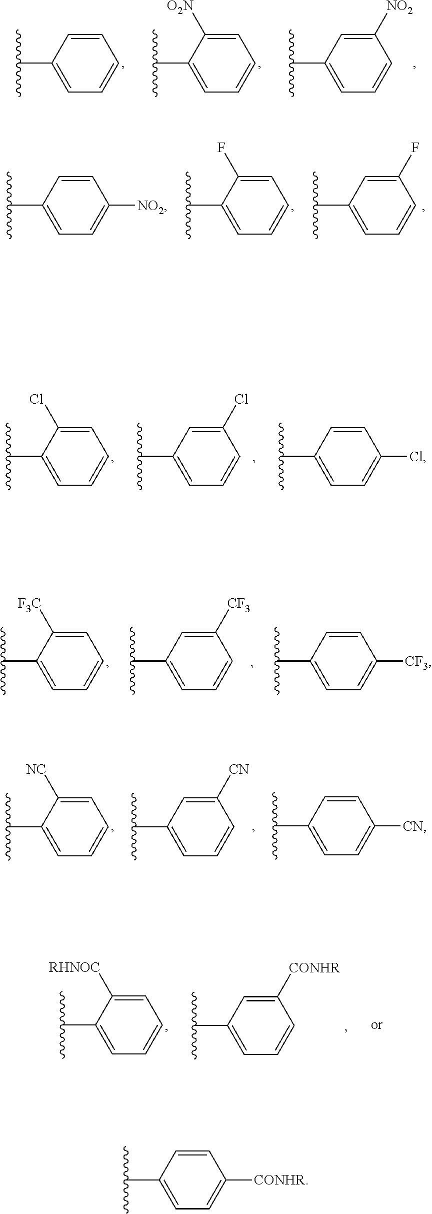

Embodiment 14 provides the compound of any one of embodiments 1-13, wherein A is

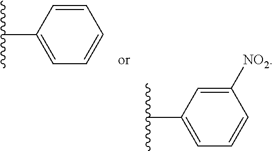 [SMILES: *c1ccccc1 ; *c1cccc([N+](=O)[O-])c1]

Embodiment 15 provides the compound which is selected from the group consisting of

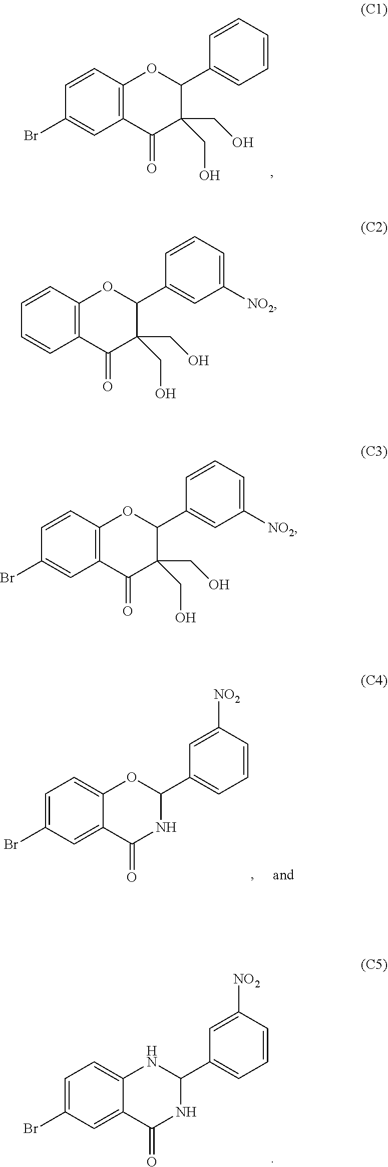 [SMILES: O=C1c2cc(Br)ccc2OC(c2ccccc2)C1(CO)CO ; O=C1c2ccccc2OC(c2cccc([N+](=O)[O-])c2)C1(CO)CO ; O=C1c2cc(Br)ccc2OC(c2cccc([N+](=O)[O-])c2)C1(CO)CO ; O=C1NC(c2cccc([N+](=O)[O-])c2)Oc2ccc(Br)cc21 ; O=C1NC(c2cccc([N+](=O)[O-])c2)Nc2ccc(Br)cc21]

Embodiment 16 provides a pharmaceutical composition comprising the compound of any one of embodiments 1-15 and at least one pharmaceutically acceptable carrier.

Embodiment 17 provides a method of treating, ameliorating, and/or preventing alcohol use disorder (AUD) in a subject, the method comprising administering to the subject a therapeutically effective amount of a compound of Formula I, or a salt, solvate, tautomer, enantiomer, diastereoisomer, and/or isotopically labeled derivative thereof:

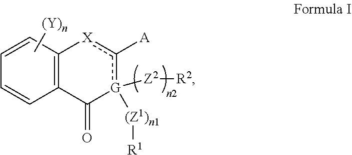

wherein:
   [symbol] is independently at each occurrence a single or double bond;
   each occurrence of Y is independently R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl;
   X is O, N, N—R, or S;
   G is C or N, wherein if G is N, then Z² and R² are absent;
   A is C₆₋₁₀ aryl or C₅₋₁₀ heteroaryl, each of which is optionally substituted by 1 to 5 substituents independently selected from the group consisting of R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂;
   Z¹ and Z² are independently at each occurrence H, O, CH₂, CHF, or CF₂;
   R¹ and R² is are each independently absent, H, OR, SR, or R;
   each occurrence of R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl;
   n is 1, 2, 3, 4, or 5;
   n1 is 1, 2, or 3; and
   n2 is 1, 2, or 3.

Embodiment 18 provides the method of embodiment 17, wherein the alcohol use disorder comprises alcoholism.

Embodiment 19 provides the method of any one of embodiments 17-18, wherein the compound is formulated as a pharmaceutical composition further including at least one pharmaceutically acceptable carrier.

Embodiment 20 provides the method of any one of embodiments 17-19, wherein the compound is

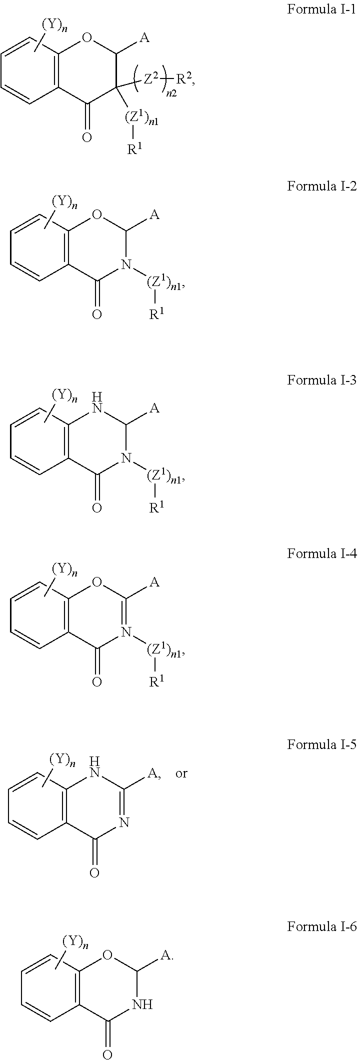

Embodiment 21 provides the method of any one of embodiments 17-20, wherein the compound is

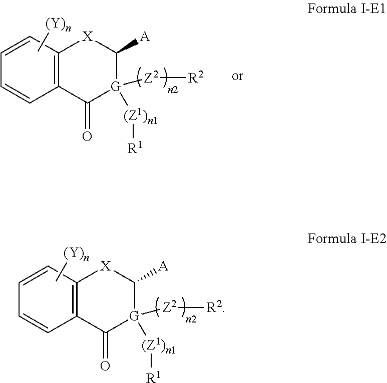

Embodiment 22 provides the method of any one of embodiments 17-21, wherein X is O.

Embodiment 23 provides the method of any one of embodiments 17-22, wherein Z¹ and Z² are CH₂.

Embodiment 24 provides the method of any one of embodiments 17-23, wherein n1 and n2 are 1.

Embodiment 25 provides the method of any one of embodiments 17-24, wherein R¹ and R² are OH.

Embodiment 26 provides the method of any one of embodiments 17-25, wherein the compound of claim 1, wherein A is

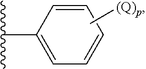

wherein
   p is an integer from 1 to 5;
   Q is selected from the group consisting of F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂; and
   R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl.

Embodiment 27 provides the method of any one of embodiments 17-26, wherein wherein A is

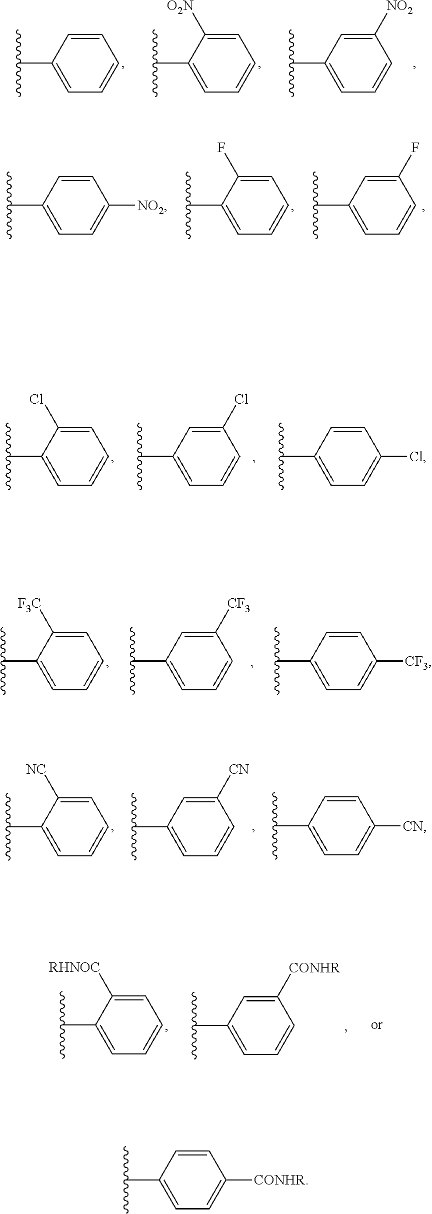

Embodiment 28 provides the method of any one of embodiments 17-27, wherein the wherein A is or

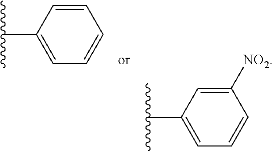 [SMILES: *c1ccccc1 ; *c1cccc([N+](=O)[O-])c1]

Embodiment 29 provides the method of any one of embodiments 17-28, wherein Y is F, Cl, Br, or I.

Embodiment 30 provides the method of any one of embodiments 17-29, wherein Y is Br.

Embodiment 31 provides the method of any one of embodiments 17-30, wherein the compound is selected from the group consisting of

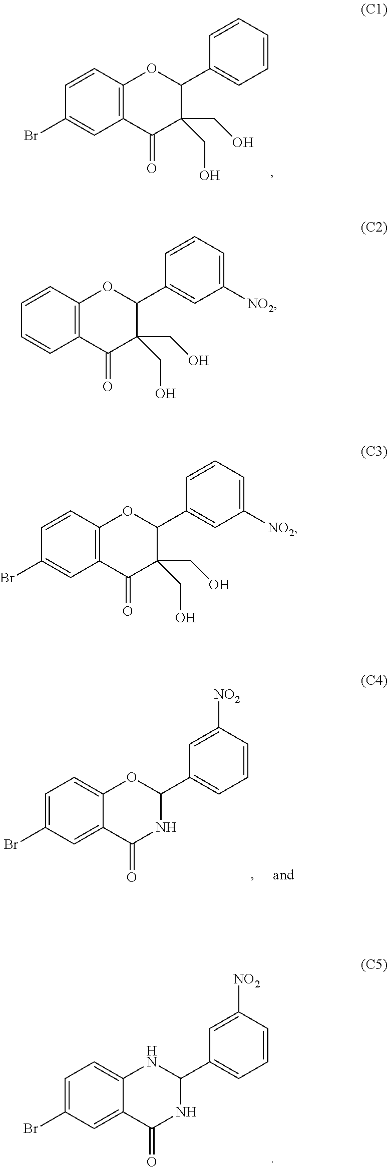 [SMILES: O=C1c2cc(Br)ccc2OC(c2ccccc2)C1(CO)CO ; O=C1c2ccccc2OC(c2cccc([N+](=O)[O-])c2)C1(CO)CO ; O=C1c2cc(Br)ccc2OC(c2cccc([N+](=O)[O-])c2)C1(CO)CO ; O=C1NC(c2cccc([N+](=O)[O-])c2)Oc2ccc(Br)cc21 ; O=C1NC(c2cccc([N+](=O)[O-])c2)Nc2ccc(Br)cc21]

Embodiment 32 provides the method of any one of embodiments 17-31, wherein the subject is a mammal.

Embodiment 33 provides the method of any one of embodiments 17-32, wherein the mammal is a human.

Embodiment 34 provides the method of any one of embodiments 17-33, wherein the subject is further administered at least one additional agent for treating, ameliorating, and/or preventing AUD.

Embodiment 35 provides the method of any one of embodiments 17-34, wherein the compound is administered by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical administration.

Embodiment 36 provides the method of any one of embodiments 17-35, wherein the route is oral administration.

1. A compound of Formula I, or a pharmaceutically acceptable salt, solvate, tautomer, enantiomer, diastereoisomer, or isotopically labeled derivative thereof: 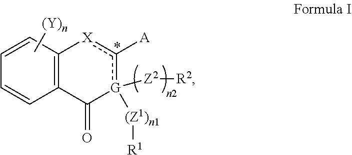
wherein: [symbol]
is independently at each occurrence a single or double bond, wherein the X—C* bond and the G-C* bond are selected such that:
if the X—C* bond is a double bond, then
the G-C* bond is a single bond, and
X is N, or
if the X—C* bond is a single bond, then
G-C* bond is a single or a double bond, and
X is O, N—R, or S;
each occurrence of Y is independently R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl;
G is C or N,
wherein if G is N or if the G-C* bond is a double bond, then Z² and R² are absent;
A is C₆₋₁₀ aryl or C₅₋₁₀ heteroaryl, each of which is optionally substituted by 1 to 5 substituents independently selected from the group consisting of R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂;
Z¹ and Z² are independently at each occurrence O, CH₂, CHF, or CF₂;
R¹ and R² are independently at each occurrence OR, SR, or R,
or —(Z¹)ₙ₁—R¹ is H,
or —(Z²)ₙ₂—R² is H;
each occurrence of R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl;
n is 1, 2, 3, 4, or 5;
n1 is 1, 2, or 3; and
n2 is 1, 2, or 3.

2. The compound of claim 1, which is:

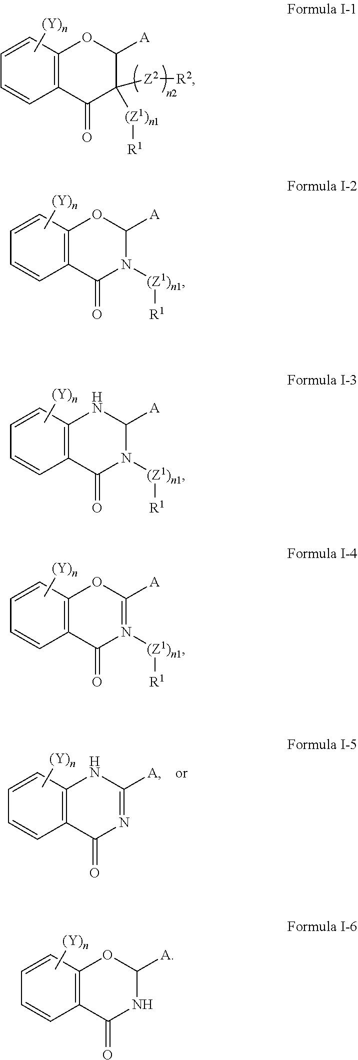

3. The compound of claim 1, which is

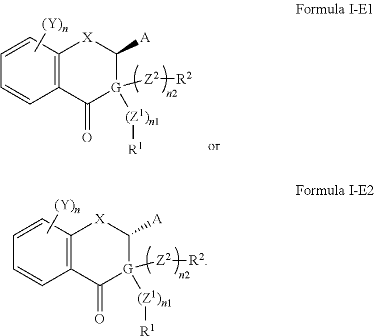

4. The compound of claim 1, wherein at least one of the following applies:
i) X is 0;
ii) G is C;
iii) Z¹ and Z² are CH₂;
iv) n1 and n2 are 1; and
v) R¹ and R² are OH.

5-8. (canceled)

9. The compound of claim 1, wherein A is 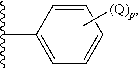
wherein
p is an integer from 1 to 5;
Q is selected from the group consisting of F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂; and
R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl.

10. The compound of claim 1, wherein Y is F, Cl, Br, or I.

11. The compound of claim 10, wherein Y is Br.

12. The compound of claim 1, wherein G is N, Z¹ is H, and R¹ is absent.

13. The compound of claim 9, wherein A is

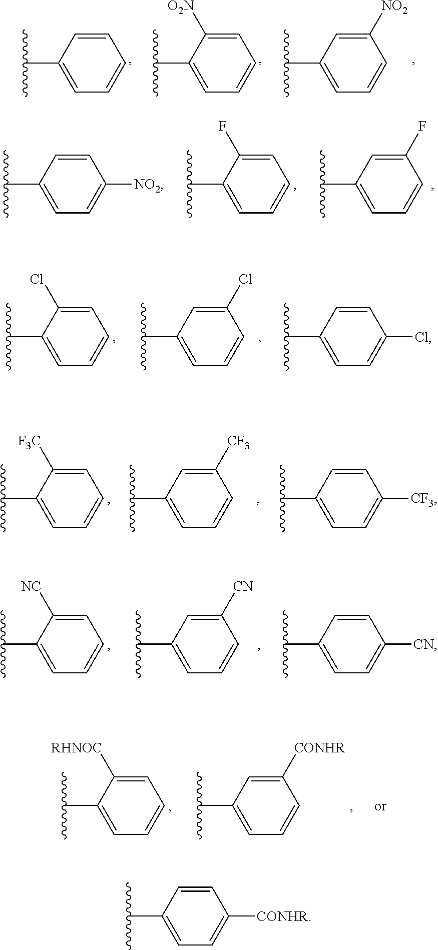

14. The compound of claim 1, wherein A is

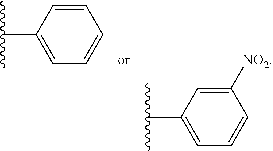 [SMILES: *c1ccccc1 ; *c1cccc([N+](=O)[O-])c1]

15. The compound of claim 1, which is selected from the group consisting of

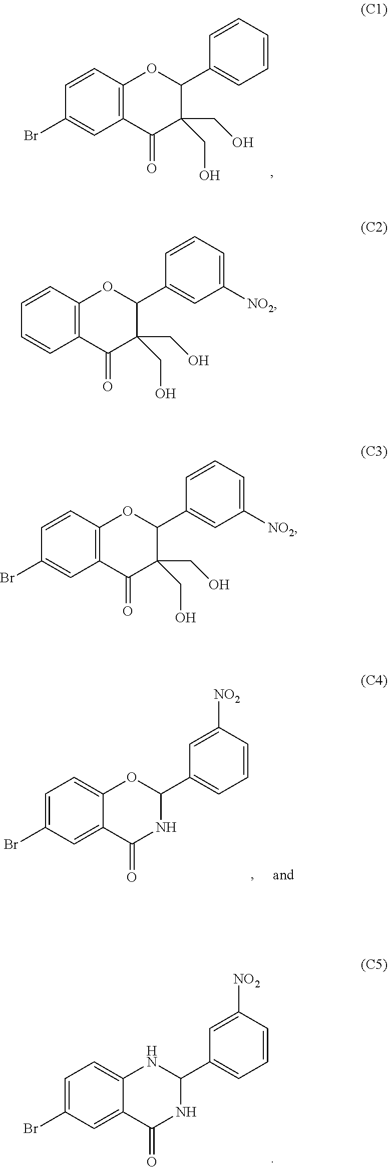 [SMILES: O=C1c2cc(Br)ccc2OC(c2ccccc2)C1(CO)CO ; O=C1c2ccccc2OC(c2cccc([N+](=O)[O-])c2)C1(CO)CO ; O=C1c2cc(Br)ccc2OC(c2cccc([N+](=O)[O-])c2)C1(CO)CO ; O=C1NC(c2cccc([N+](=O)[O-])c2)Oc2ccc(Br)cc21 ; O=C1NC(c2cccc([N+](=O)[O-])c2)Nc2ccc(Br)cc21]

16. A pharmaceutical composition comprising the compound of claim 1 and at least one pharmaceutically acceptable carrier.

17. A method of treating or ameliorating alcohol use disorder (AUD) in a subject in need thereof, the method comprising:
administering to the subject a therapeutically effective amount of a compound of Formula I, or a pharmaceutically acceptable salt, solvate, tautomer, enantiomer, diastereoisomer, or isotopically labeled derivative thereof: 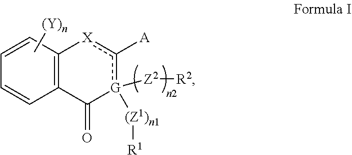
wherein: [symbol]
is independently at each occurrence a single or double bond, wherein the X—C* bond and the G-C* bond are selected such that:
if the X—C* bond is a double bond, then
the G-C* bond is a single bond, and
X is N, or
if the X—C* bond is a single bond, then
G-C* bond is a single or a double bond, and
X is O, N—R, or S;
each occurrence of Y is independently R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, OC(═O)N(R)₂, C₃₋₁₀ heterocycloalkyl, and C₅₋₁₀ heteroaryl;
G is C or N,
wherein if G is N or if the G-C* bond is a double bond, then Z² and R² are absent;
A is C₆₋₁₀ aryl or C₅₋₁₀ heteroaryl, each of which is optionally substituted by 1 to 5 substituents independently selected from the group consisting of R, F, Cl, Br, I, OR, CN, NO₂, N(R)₂, SR, S(═O)R, SF₃, SF₅, S(═O)₂R, S(═O)₂N(R)₂, P(═O)OH₂, P(═O)OR₂, C(═O)R, C(═O)OR, C(═O)N(R)₂, OC(═O)R, and OC(═O)N(R)₂;
Z¹ and Z² are independently at each occurrence O, CH₂, CHF, or CF₂;
R¹ and R² are independently at each occurrence OR, SR, or R,
or —(Z¹)ₙ₁—R¹ is H,
or —(Z²)ₙ₂—R² is H;
each occurrence of R is independently selected from the group consisting of hydrogen, C₁₋₆ alkyl, C₂₋₆ alkenyl, C₂₋₆ alkynyl, C₁₋₆ haloalkyl, C₃₋₈ cycloalkyl, C₃₋₈ cycloalkenyl, and C₃₋₈ halocycloalkyl;
n is 1, 2, 3, 4, or 5;
n1 is 1, 2, or 3; and
n2 is 1, 2, or 3.

18. The method of claim 17, wherein the alcohol use disorder comprises alcoholism.

19. The method of claim 17, wherein the compound is formulated as a pharmaceutical composition further including at least one pharmaceutically acceptable carrier.

20. The method of claim 17, which is selected from the group consisting of

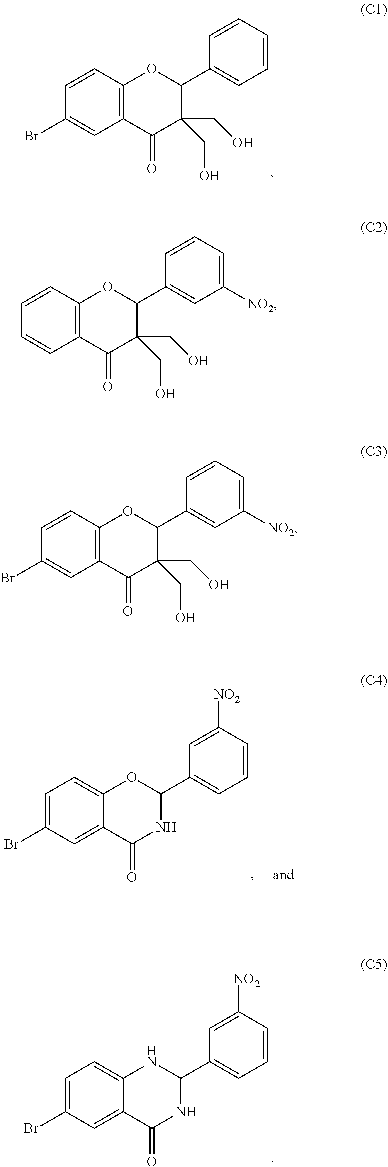 [SMILES: O=C1c2cc(Br)ccc2OC(c2ccccc2)C1(CO)CO ; O=C1c2ccccc2OC(c2cccc([N+](=O)[O-])c2)C1(CO)CO ; O=C1c2cc(Br)ccc2OC(c2cccc([N+](=O)[O-])c2)C1(CO)CO ; O=C1NC(c2cccc([N+](=O)[O-])c2)Oc2ccc(Br)cc21 ; O=C1NC(c2cccc([N+](=O)[O-])c2)Nc2ccc(Br)cc21]

21. The method of claim 17, wherein the subject is human.

22. (canceled)

23. The method of claim 17, wherein the subject is further administered at least one additional agent for treating or ameliorating AUD.

24. The method of claim 17, wherein the compound is administered by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical administration.

25. The method of claim 24, wherein the route is oral administration.
